SEQUENCE LISTING (3 sequences)

SEQ ID NO: 1 - DNA, 26 bp, Artificial Sequence
  Oligonucleotide
gannnngcnggngayggnacnacnac

SEQ ID NO: 2 - DNA, 26 bp, Artificial Sequence
  Oligonucleotide
yknykntcnccraanccnggngcytt

SEQ ID NO: 3 - DNA, 28 bp, Artificial Sequence
  Oligonucleotide
gacaaagtcggtaaagaaggcgttatca

(19) United States
(12) Patent Application Publication
(10) Pub. No.: US 2004/0241662 A1
(43) Pub. Date: Dec. 2, 2004

(54) Detecting microbial contamination in grain and related products

(76) Inventors: W. Wade Robey, Excelsior, MN (US); Alison M. Jones, Eden Prairie, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/449,458

(22) Filed: May 30, 2003

(51) Int. Cl. A61K 39/38; C12Q 1/68; G01N 33/53; A61K 39/00

(52) U.S. Cl. 435/6

(57) ABSTRACT

The invention provides for methods of determining the presence, absence, or amount of microbial contamination in grain and related products. The invention further provides for methods of monitoring grain and related products before, during, or after processing of the grain or related product into, for example, feed. The invention also provides for articles of manufacture for carrying out the claimed methods.

### TECHNICAL FIELD

This invention relates to grain and related products, and more particularly to methods of detecting microbial contamination in grain and related products.

### BACKGROUND

Toxic metabolic by-products of fungi, known as mycotoxins, have received considerable attention during the past several years. Mycotoxins are known to cause serious health problems in animals including equine leukoencephalomalacia in horses and porcine edema in swine. Reduced weight gain, capillary fragility, reduced fertility, reduced immunocompetence, and even death have been attributed to mycotoxins. No animal is known to be resistant, but in general, older animals are more tolerant than younger animals. Some mycotoxins, fumonisin, aflatoxin, and ochratoxin in particular, also have been associated with human health problems. Certain mycotoxins are suspected carcinogens.

Most mycotoxins of concern are produced by three genera of fungi, Aspergillus, Penicillium, and Fusarium. These organisms are not aggressive pathogens, but instead thrive on pre-harvest and post-harvest substrates. Several factors influence mycotoxin contamination of stored grain by affecting fungal growth and metabolism. These factors include water activity, substrate aeration and temperature, fungal load, insect and/or mechanical damage of grain, etc.

Thus, methods for detecting microbial contamination in grain or in a related product are useful so that illness or disease can be avoided in humans and animals that consume the grain or related products.

### SUMMARY

The invention provides for methods of determining the presence, absence, or amount of microbial contamination in grain and related products such as those consumed by animals and humans. The invention further provides for methods of monitoring grain and related products before, during, or after processing.

In one aspect, the invention provides for a method of determining the presence, absence, or amount of microbial contamination in grain and related products. The method includes the steps of providing a sample from the grain or related product, and determining the presence, absence, or amount of microbial contamination in the sample. Typically, the presence of microbial contamination in the sample indicates the presence of microbial contamination in the grain or related product. The microbial contamination can be by one or a few species or genera of microbes, or can be by many species or genera of microbes.

In one embodiment, the microbes are taxonomically and phylogenetically identified. Generally, microbial contamination can include microbes that are bacteria, fungi, viruses, or protozoa. Representative bacterial microbes include those from the Staphylococcus, Streptococcus, Pseudomonas, Escherichia, Bacillus, Brucella, Chlamydia, Clostridium, Shigella, Mycobacterium, Agrobacterium, Bartonella, Borellia, Bradyrhizobium, Ehrlichia, Haemophilus, Helicobacter, Heliobacter, Lactobacillus, Neisseria, Rhizobium, Streptomyces, Synechococcus, Zymomonas, Synechocyotis, Mycoplasma, Yersinia, Vibrio, Burkholderia, Franciscella, Legionella, Salmonella, Bifidobacterium, Enterococcus, Enterobacter, Citrobacter, Bacteroides, Prevotella, Xanthomonas, Xylella, and Campylobacter genera; representative fungal microbes include those from the Aspergillus, Fusarium, Penicillium, Claviceps, Colletrotrichum, Cochliobolus, Helminthosporium, Microcyclus, Puccinia, Pyricularia, Deuterophoma, Monilia, Candida, and Saccharomyces genera; representative viral microbes include those from the Coronaviridae genus; and representative protozoan microbes include those from the Acanthamoeba, Cryptosporidium, and Tetrahymena genera.

Generally, grains include, but are not limited to, corn, wheat, soybean, barley, sorghum, and rice. A sample used in the methods of the invention can be obtained from any of the above-listed grains. Grains can be processed into related products for human consumption or for animal consumption. Representative products for human consumption include, for example, rice, breads, muffins, cakes, cereal, pasta, and dough. Processed grains also can be used directly in feed products for animal consumption. A sample used in the methods of the invention can be obtained from any of the above-listed processed grains or related products. A single sample can be obtained, or a plurality of samples can be obtained. In one embodiment, the plurality of samples can be pooled.

Microbial contamination can be detected using a number of methods. For example, the determining step can include microbial culturing and colony identification, genetic fingerprinting, ribosomal genotyping, and/or cpn60 genotyping. In general, the method used to determine the presence, absence, or amount of microbial contamination can be specific for one or a few species or genera of microbes or can be universal for many species or genera of microbes.

Methods of the invention can also include providing a control sample, and determining the amount of microbes in the control sample. Such a control sample can be a known amount of microbes or of a particular species of microbe.

In another aspect, the invention provides a method for monitoring grain or a related product before, during, or after processing of the grain or related product. Such a method includes providing a sample from the grain or related product, determining the presence, absence, or amount of microbial contamination in the sample, and tailoring the processing based upon the presence, absence, or amount of the microbial contamination. For example, when the sample is obtained before the processing of the grain or related product, the tailoring step includes abandoning the processing, or expediting the processing. For example, when the sample is obtained during the processing of the grain or related product, the tailoring step includes abandoning the processing, expediting the processing, or repeating all or a portion of the processing. For example, when the sample is obtained after the processing of the grain or related product, the tailoring step can include repeating all or a portion of the processing. The tailoring step can be based on the presence or absence of microbial contamination, the amount of the microbial contamination, or both.

In another aspect, the invention provides methods for monitoring grain or animal feed. Such methods include providing a sample from the grain or feed; determining the presence, absence, or amount of microbial contamination in the sample; and tailoring treatment of the grain or animal feed based upon the presence, absence, or amount of microbial contamination.

In embodiments of the invention, the treatment can include blending the grain or feed; feeding select animals with the animal feed; applying a mycotoxin binder to the animal feed; and/or applying an acid-based fungal inhibitor to the grain.

In yet another aspect of the invention, there is provided an article of manufacture that includes at least one microbial antibody that is attached to a solid support, and instructions for collecting a sample from grain or related products and determining the presence, absence, or amount of microbial contamination in the sample. The article of manufacture can further include an indicator molecule. In one specific embodiment, the solid support is a dipstick.

In still another aspect of the invention, there is provided an article of manufacture that includes at least one oligonucleotide that is complementary to nucleic acid sequences from one or a few number of microbial species; and instructions for collecting a sample from grain or a related product and for determining the presence, absence, or amount of microbial contamination in the sample. Alternatively, an article of manufacture of the invention can include at least one oligonucleotide that is complementary to nucleic acid sequences from microbial species of at least two genera and the above-described instructions.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DETAILED DESCRIPTION

The invention provides for methods for determining the presence, absence, or amount of microbial contamination in grain or in a related product. Grains include, without limitation, corn, soybean, wheat, barley, sorghum, and rice. Grains can be processed into a variety of related products, many of which are consumed by humans and/or animals. In one respect, grain can be processed into products for human consumption such as rice, breads, muffins, cakes, cereals, pasta, and dough. In another respect, grain is processed for use as animal feed, wherein the processing breaks down the endosperm so that the animals utilize the starch more efficiently. Therefore, the processing of grain into feed increases the energy value. The invention further provides for methods of monitoring grain or related products before, during, or after processing of the grain into feed or into a related product.

Mycotoxins

Mycotoxins, a by-product of fungi, are a major problem during grain storage and processing. Aflatoxins, metabolites of the fungus Aspergillus flavus, A. parasiticus and A. nomis are potent liver toxins and carcinogens in animals. Aspergillus flavus is common in corn and cottonseed mill. A. parasiticus is more common in peanuts. Typically, the fungus has a yellow green appearance when it is growing on corn kernels. The fungus is quite common in nature, but its population increases during hot dry weather. Aflatoxin contamination is greater in corn that has been produced under stress conditions. Thus, drought, heat, insect, nematode, and fertilizer stress are all conducive to high levels of aflatoxins. Management practices such as irrigation, good insect control and timely fertilization may reduce stress to the corn plant and thus lower aflatoxin levels.

Fumonisins are a group of mycotoxins produced by fungi in the genus Fusarium. The fungus Fusarium moniliforme (Fusarium verticillioides) is a common pathogen of corn. Fusarium moniliforme usually appears white to salmon colored, although it may not be visible on the corn kernel. This fungus often produces a symptom on the corn kernels referred to as “starburst,” or a white streaking of the kernel. The visual absence of mold, however, does not mean that kernels do not contain the toxin. Intact corn kernels may contain the fungus and the toxin but show no sign of the fungal contamination. In severe cases, the corn shucks will become “glued” to the kernels in the cob. Fumonisins have been implicated as a possible cause of human esophageal cancer, equine leukoencephalomalacia (ELEM), a serious disease in horses, and porcine edema, a disease in swine.

Deoxynivalenol (DON or vomitoxin) is a mycotoxin produced by certain species of Fusarium, the most important of which is F. graminearum (Gibberella zeae). This fungus causes Gibberella ear (also known as red ear rot) or stalk rot on corn and head scab in wheat. The fungus itself appears reddish to pinkish. The fungus may cause a reddish discoloration of the cob and kernels. Red ear rot caused by F. graminearum is favored by warm wet weather after silking. Disease tends to be worse when corn is grown without rotation or after wheat as this pathogen also infects wheat. It may be worse when corn is grown in reduced tillage situations. The mycotoxin deoxynivalenol causes reduced weight gain and suppresses animal feeding, especially in swine. At high concentrations (greater than 10 ppm), vomiting and total feed refusal may occur.

Zearalonone is a Fusarium produced mycotoxin that elicits an estrogenic response in monogastrics. Usually, zearalonone is found secondary to and at much lower levels than DON. Ruminants are able to ruminally degrade zearalonone, and therefore, zearalonone is less toxic to dairy cattle. Field observations of poor feed intake, depressed milk production, and reproductive problems have been associated with the presence of DON and zearalonone. This may be attributed to the interaction of mycotoxins with other factors or other mycotoxins.

Ochratoxin A (OA) is a mycotoxin produced by certain species of Aspergillus and Penicillium. OA has been detected in a wide range of foods from both temperate regions (e.g., European cereals) and tropical climates (e.g., coffee, cocoa). OA was classified as a genotoxic carcinogen by the European Scientific Committee for Food. OA can be found in raw materials such as cereals and green coffee. OA is very stable and can survive processing, and therefore can be found in cereal products, beer, and roasted coffee. OA also has been found in dried fruit, nuts, pulses, grape juice, wine, cocoa, and pork products.

T-2 toxin is another Fusarium-produced mycotoxin and is seldom detected in forages of the upper Midwest of the U.S. Studies indicate that T-2 causes gastroenteritis in laboratory animals. Studies in cattle indicate an association between the toxin and feed refusal and diarrhea, as well as immunosupression in dairy calves.

Ergot is a fungus that can infect grain that has suffered damage from the elements. Ergot alkaloids are produced by the fungal parasites Claviceps purpurea and Claviceps paspali. The growth of these fungal parasites is prompted by warm moist conditions. These types of ergot alkaloids that infect grain will vary with the region, season, and grain type. Ergot is usually seen quite easily as dark, banana shaped masses and is usually slightly larger than the grain kernels. The effects of ergot when consumed by livestock include gangrene of the distal extremities (feet, ears, tail), derangement of the central nervous system, and failed milk production. Spontaneous abortions have also been linked to ergot consumption. In extreme cases, livestock can die from consuming high levels of ergot-contaminated feed.

Analytical test methods are available to detect the presence of mycotoxins in a sample. Such methods are not ideal due to difficulty in sampling, turnaround time of the method, and cost of the method. The methods described herein for detecting microbial contamination can be coupled with an analytical method for detecting mycotoxins (e.g., ELISA) to confirm the presence of mycotoxin-producing fungi. If the methods described herein also identify the particular fungus present in grain, then an analytical method specific for the appropriate mycotoxin can be used. The methods of the invention can be used to certify that grain, feed, or a related product is mold- or toxin-free.

Animal Feed

Grain also can be processed for use as animal feed. Grain processing methods are used to rupture the protective seed coat, increase surface area, gelatinize the starch, or reduce interference of the endosperm protein matrix with digestion of the starch fraction. Grain processing methods used mechanical action, moisture, or heat, either singly or in combination. The following are brief descriptions of grain processing methods.

Grinding: This is usually done with a hammermill. Factors influencing the fineness of the end product include screen size, hammermill size, power and speed, type of grain and moisture content of grain.

Dry Rolling: Grain is passed through rollers that are typically grooved on the surface. Particle size varies from very small to very coarse and is influenced by roller weight, size of grooves, pressure and spacing, moisture content of the grain and rate of grain flow.

Steam Rolling: Grain is exposed to steam for one to eight minutes prior to rolling. Sometimes this processing is also known as crimping or steam crimping. The steam softens the kernel, producing a more intact, crimped-appearing product than dry rolling. The moisture content of the grain is also increased slightly.

Pelleting: Grain is usually ground or rolled before processing through a pellet mill. The primary advantage of this method is in the mechanization of feed handling.

Steam Flaking: Grain is subjected to steam under atmospheric conditions for usually 15 to 30 minutes, before rolling. Large, heavy roller mills set at near zero tolerance produce a very thin, flat flake which usually weighs from 22 to 28 pounds per bushel and contains 16 to 20 percent moisture. The flaking process causes gelatinization of the starch granules (hydration or rupturing of the starch molecule) rendering the starch more digestible. The degree of flaking and level of gelatinization appear to be influenced by such factors as steaming time, temperature, grain moisture, roller size and tolerance, processing rate, and type and variety of grain.

Pressure Flaking: The grain is subjected to steam under pressure for a short time, such as 50 pounds per square inch for one to two minutes. A continuous flow cooker is operated by air-lock valves to inject and eject grain. Steam is injected into the cooker at the desired pressure. The grain in the chamber reaches a temperature approaching 300° Fahrenheit. When the grain is expelled from the cooker, it is generally cooled (by use of a cooling and drying tower) to below 200° F. and 20% moisture before flaking.

High-Moisture Harvesting: Grain is harvested and stored at 20 to 30% moisture. Cereal grains are physiologically mature when the moisture content of the grain drops below 38 to 40%. High moisture harvested grain may be ground and stored in a horizontal silo or in the whole form if oxygen-limited storage is used. Whole grain cannot be packed adequately to avoid substantial spoilage in horizontal silos.

Reconstituting: Dry grain is reconstituted to 25 to 30% moisture and stored whole in oxygen-limited conditions for 10 to 20 days before feeding.

Chemical Preservation: Acids, usually propionic or a combination of propionic and acetic, are added to moist, early-harvested grain to permit storage of the whole kernel wet grain in conventional facilities, such as wooden bins or trench silos. The wetter the grain, the more acid is required for satisfactory preservation; thus, the expense of preservation increases with the increase in moisture content of the grain.

Popping: Air-dry grain with a moisture content of 10 to 14% is popped by heating it with high temperature air at 700-800° F. for 15 to 30 seconds. Not all of the grain pops, but the resulting product very much resembles ordinary popcorn and has a moisture content of approximately 3%. Popping causes disruption of the starch granules by using natural moisture in the kernel to steam, gelatinize and expand the starch granules. Rolling and remoisturization are usually essential.

Micronizing: Dry grain that has been heated with gas-fired, infrared generators as the grain passes along an oscillating steel plate or skillet and then dropped into Knorling rolls has been termed micronized grain. The term was coined from the word microwave (microwaves are emitted from the infrared generators) and from the unique type of rollers used. The grain is discharged at the end of the plate at about 300° F. The rolls have a spiral groove which places high diagonal and parallel pressure on the grain. The product has an intact, flake-like appearance, very much resembling some steam-flaked grains. Temperature of the grain and density of the final product can be regulated.

Exploding: This technique involves delivering raw grain into high tensile strength steel “bottles” which hold approximately 200 pounds of grain. Live steam is injected into the bottles until pressure reaches 250 pounds per square inch. After about 20 seconds, a valve opens to let the grain escape as expanded balls with the hulls removed. Under the high pressure, moisture is forced into the kernels, which, when released into the air, swell to several times their original size.

Extruding: Extruded grain is produced by a machine which applies heat and pressure by means of friction as the grain passes through a tapered screw. Dry whole grain, with no moisture added, is crushed by an auger-like rotor and forced through an orifice, producing “ribbons” which break into flakes about 1/32 inch in thickness.

Roasting: Grain is passed through a roaster. The grain is heated to about 300° F. and has a roasted odor with an oily, puffed and slightly caramelized appearance.

Cereal Grains and Related Products

Types of Products

Cereal grains and related products include baked goods (breads, muffins, cakes, pastries, cookies, biscuits, bagels, and so on), frozen and refrigerated dough, breakfast cereals (cold cereal, oatmeal, grits, and so on), refrigerated or dry pasta and noodles, and cooked grains (for example, rice). Some products, such as baked goods, have a long history of safe storage at room temperature; others, such as rice, require time/temperature control after preparation.

Pathogens of Concern

Grains and milled products are raw agricultural commodities; therefore, a variety of microorganisms, including mold, yeast, coliforms and other bacteria, occur naturally. Grains and milled products are dried to inhibit mold growth during storage, a process that easily controls growth of bacterial pathogens. Therefore, while organisms such as Salmonella spp. may be present, the prevalence and levels are low (usually >1%). Raw ingredients used to prepare dough products (for example, eggs, dairy products, meats) may introduce Salmonella spp., and need to be considered when analyzing potential hazards. Staphylococcus aureus may present a potential hazard for certain raw dough, such as dough processed at warm temperatures for extended periods of time (days); however, yeast leavened dough control the organism thorough competitive inhibition. Bacillus cereus presents a concern in cooked rice.

Effects of Processing

Baking, boiling, steaming, or frying are the methods used to cook the cereal-grain products. The temperatures required to achieve product quality usually destroy vegetative pathogens that may be present. These temperatures are needed to properly set the starch structure and/or to rehydrate dry products. Baking and frying not only destroy vegetative pathogens such as S. aureus and Salmonella spp., but they also remove moisture from the product, especially at the exterior surface. This dehydrated surface inhibits the growth of most bacteria; thus, mold is the primary microbial mode of failure for baked goods. When stored at room temperature, baked and fried products typically continue to lose moisture to the atmosphere, further reducing the potential for pathogen growth. Thus, baked and fried cereal-grain products such as cakes, breads, muffins, and biscuits have a long history of safe storage at room temperature.

While boiled or steamed cereal products achieve temperatures lethal to vegetative pathogens during the cooking process, these products increase in water activity (i.e., moisture content; a_{w}) to levels that support the growth of many microbial pathogens. Thus, time/temperature control is required to assure the safety of these products. For example, numerous B. cereus outbreaks have been associated with fried rice prepared using boiled rice that was held for hours at room temperature.

Time/temperature Control

Although baked and fried cereal-grain products (for example, cakes, breads, muffins, and biscuits) have a high a_{w}, a number of reasons may justify their shelf-stability: they have a long history of safe storage at ambient temperature; processing temperatures and moisture reduction, especially on the surface, preclude the growth of pathogens; and they are often formulated to include ingredients that enhance product safety and stability so as to permit distribution without temperature control for limited periods of time. Ingredients that are used to enhance safety and stability include humectants to reduce a_{w} (sugars and glycerine), preservatives (calcium propionate, potassium sorbate, sorbic acid), acids to reduce pH (vinegar, citric acid, phosphoric acid, malic acid, fumaric acid), spices with antimicrobial properties (cinnamon, nutmeg, garlic), and water-binding agents to control free water (gums, starches). The primary mode of spoilage of baked goods is mold growth.

Dough is frequently used to enrobe other food ingredients. Careful consideration must be given to these combination products to accurately assess the need for time/temperature control. For example, egg and dairy ingredients baked inside a pastry, such as cream-cheese croissant, will receive sufficient heat treatments to destroy vegetative pathogens and may therefore be stable at room temperature with water activities above 0.86. However, if the filing is injected after the baking process, as in the case of cream-filled eclair, the potential for contamination must be assessed. Meat and vegetable-filled cereal products with high water activities (>0.94) and neutral pH generally require time/temperature control because the baking process can activate spore formers such as C. botulinum that are present in these ingredients.

The processing of grain into feed, or treatment of the grain or feed itself, can be tailored based on the presence, absence, or amount of fungi detected in a sample. The presence of fungi itself is detrimental to the grain or feed, as fungi use the protein and carbohydrates as an energy source and can significantly alter the nutrient profile of the grain or feed. The presence of mycotoxin-producing fungi, however, is a health hazard to humans or animals consuming such contaminated grain or feed. The particular treatment of the grain or feed can be tailored depending upon the particular fungi and/or mycotoxin present and their respective levels. For example, contaminated grain or feed can be blended with uncontaminated grain or feed, respectively, to reduce the level of contamination. In addition, depending upon the level of fungal or mycotoxin contamination in feed, a decision can be made as to the use of the feed (e.g., not using such feed in certain populations of animals such as breeder stocks or starter animals). A binder also can be applied to the feed to bind mycotoxin. Binders such as Agrabond (Agranco Corp., Coral Gables, Fla.) or Toxifarm® Dry (Farmavet International, Istanbul, Turkey) are clay-based products used routinely by those in the art to remove mycotoxin from feed. Based on identification of the contaminating fungus, a binder specific for the particular mycotoxin can be applied to the feed. Further, grain can be treated with an acid-based fungal inhibitor (e.g., containing, for example, propionic acid, sorbic acid, acetic acid, phosphoric acid, or combinations thereof) to arrest further fungal growth. Such tailoring as described herein can be used to achieve optimal use of the grain or feed while reducing any health hazard posed by contaminating fungi.

The invention provides for methods of monitoring grain and related products before, during, or after processing. Depending upon the presence, absence, or amount of microbial contamination, the processing step can be tailored to address the presence and amount of microbial contamination. For example, if a sample is obtained from grain or a related product that has not been processed and the presence of contaminating microbes is detected, the grain or related product can be processed expeditiously if, for example, high amounts of microbial contamination are detected, or not processed at all (e.g., discarded) if a high amount of microbial contamination is detected. If a sample is obtained during processing of the grain or related product and microbial contamination is detected, the grain or related product can be processed expeditiously, not processed at all, or all or a portion of the processing can be repeated. If a sample is obtained from a processed grain or related product and microbial contamination is detected, the processing can be repeated in part or in full.

Samples and Sampling Methods

The methods described herein are capable of detecting the presence, absence, or amount of a microbe in a sample obtained from grain and related products. Methods of the invention also can be used for monitoring grain or a related product before, during, and after processing. The presence or absence of microbes can be determined in a sample from grain or a related product. As used herein, “biological sample” refers to any sample obtained, directly or indirectly, from grain or a related product, before, during, or after being processed. Therefore, a sample useful in the methods of the invention includes any portion of the grain or related product, processed or otherwise, listed above. Without limitation, samples can be grain samples, or portions of a processed grain or a related product (e.g., samples of rice or pasta, or a portion of a muffin, or a cake).

Proper sampling is the most important step in determining contamination levels. Contaminated kernels are usually not uniformly distributed in a load. This makes it possible for one sample to contain a probe-full from a hot spot while the next contains no contaminated kernels. Sampling grain and related products can include taking a single sample or taking multiple samples. Multiple samples can be pooled and analyzed together, or can be kept separate and analyzed individually. Since microbial contamination may not be uniform throughout the grain or the related product, it may be advantageous to obtain multiple samples using, for example, a systematic and/or stratified sampling arrangement (see, for example, Thompson, 1997, Ciba. Found. Symp., 210:161-72; and Park & Pohland, 1989, J Assoc. Off Anal. Chem., 72(3):399-404). The American Society for Testing and Materials (ASTM) publishes standards for various sampling methods. See, for example, ASTM Standards on Environmental Sampling, 2^{nd} Ed., 1997; ASTM Standards on Environmental Site Characterization, 2^{nd}, Ed., 2002; and ASTM Standard Practice for Aseptic Sampling of Biological Materials, 1999. In addition, resources are available that provide guidance in ensuring appropriate sample design and statistical analysis of results (see, for example, Anderson-Sprecher et al., 1994, J. Expo. Anal. Environ. Epidemiol., 4(2):115-31; and ASTM Standard Practice for Probability Sampling of Materials, 1996).

Methods for sampling with a swab are known to those of skill in the art. Generally, a swab is hydrated (e.g., with an appropriate buffer, such as Cary-Blair medium, Stuart's medium, Amie's medium, PBS, buffered glycerol saline, or water) and used to sample for a microbe. Any microbe present is then recovered from the swab, such as by centrifugation of the hydrating fluid away from the swab, removal of supernatant, and resuspension of the centrifugate in an appropriate buffer, or by washing of the swab with additional diluent or buffer. The so-recovered sample may then be analyzed according to the methods described herein for the presence of a microbe. Alternatively, the swab may be used to culture a liquid or plate (e.g., agar) medium in order to promote the growth of any microbes for later testing. Suitable swabs include both cotton and sponge swabs; see, for example, those provided by Tecra®, such as the Tecra ENVIROSWAB®.

Methods for collecting and storing samples are generally known to those of skill in the art. For example, the Association of Analytical Communities International (AOAC International) publishes and validates sampling techniques for testing foods and agricultural products for microbial contamination. See also WO 98/32020 and U.S. Pat. No. 5,624,810, which set forth methods and devices for collecting and concentrating microbes.

In particular embodiments of the methods described herein, a separation and/or concentration step may be necessary to separate any microbes present from other components of a sample or to concentrate the microbe to an amount sufficient for rapid detection. For example, a sample suspected of containing a biological microbe may require a selective enrichment of the microbe (e.g., by culturing in appropriate media, e.g., for 4-96 hours, or longer) prior to employing the detection methods described herein. Alternatively, appropriate filters and/or immunomagnetic separations can concentrate a microbe without the need for an extended growth stage. For example, antibodies with binding affinity to a microbial polypeptide can be attached to magnetic beads and/or particles. Multiplexed separations, in which two or more processes for concentration are employed, are also contemplated (e.g., centrifugation, membrane filtration, electrophoresis, ion exchange, affinity chromatography, and immunomagnetic separations).

The samples from the grain or related product can be used “as is,” or may need to be treated prior to application of the detection methods employed herein. For example, samples can be processed (e.g., by nucleic acid or protein extraction methods and/or kits known in the art) to release nucleic acid or proteins. In other cases, a biological sample can be contacted directly with PCR reaction components and appropriate oligonucleotide primers and probes.

Detection of Microbial Contamination

As used herein, “microbial contamination” refers to the presence of one or more bacteria, protozoa, viruses, and/or fungi. Microbial contamination is not limited to the presence of pathogenic microbes. Microbes that can contaminate grain and related products include the following examples of prokaryotic genera: Staphylococcus, Streptococcus, Pseudomonas, Escherichia, Bacillus, Brucella, Chlamydia, Clostridium, Shigella, Mycobacterium, Agrobacterium, Bartonella, Borellia, Bradyrhizobium, Ehrlichia, Haemophilus, Helicobacter, Heliobacter, Lactobacillus, Neisseria, Rhizobium, Streptomyces, Synechococcus, Zymomonas, Synechocyotis, Mycoplasma, Yersinia, Vibrio, Burkholderia, Franciscella, Legionella, Salmonella, Bifidobacterium, Enterococcus, Enterobacter, Citrobacter, Bacteroides, Prevotella, Xanthomonas, Xylella, and Campylobacter; the following examples of protozoa genera: Acanthamoeba, Cryptosporidium, and Tetrahymena; the following examples of fungal genera: Aspergillus, Colletrotrichum, Cochliobolus, Helminthosporium, Microcyclus, Puccinia, Pyricularia, Deuterophoma, Monilia, Candida, and Saccharomyces; and the following viral microbes: Coronaviridae.

The microbes in a sample can be evaluated and monitored using a number of methods. For example, the microbes in a sample can be cultured and colonies identified and/or enumerated. It has been estimated, however, that culturing typically recovers only about 0.1% of the microbial species in a sample (based on comparisons between direct microscopic counts and recovered colony-forming units). An improvement on culture-based methods is a community-level physiological profile. Such determinations can be accomplished by monitoring the capacity of a microbial community to utilize a suite of carbon sources with subsequent detection of the end product of this carbon metabolism by, for example, reduction of a tetrazolium dye. Profiling the physiology of a microbial community can yield qualitative (e.g., different patterns of reduced substrates) and semi-quantitative (e.g., spectrophotometric measurement of reduction) results. Biolog, Inc. (Hayward, Calif.) has commercialized a microtiter plate assay useful for determining the physiological profile of a complex microbial community. The BIOLOG method requires a standard inoculum density of metabolically active microorganisms, and assumes that all members of the community grow at the same rate so the utilization profile is not skewed by the metabolic capabilities of the fastest growers, and further assumes that the 95 substrates reflect the comprehensive substrate availability in the environment of interest.

Culture-independent methods to evaluate the microbes in a sample consist of extracting and analyzing microbial macromolecules from a sample. In general, useful target molecules are ones that, as a class, are found in all microorganisms, but are diverse in their structures, thereby reflecting the diversity of the microbes. Examples of target molecules include phospholipid fatty acids (PLFA), polypeptides, and nucleic acids. PLFA analysis is based on the universal presence of modified fatty acids in microbial membranes, and is useful as a taxonomic tool. PLFAs are easily extracted from samples, and separation of the various signature structures reveals the presence and abundance of classes of microbes. This method requires appropriate signature molecules, which often are not known or may not be available for the microbes of interest. In addition, the method requires that an organism's PLFA content does not change under different metabolic conditions. Another limitation to using PLFAs as target molecules is that widely divergent organisms may have the same signature set of PLFAs.

Various nucleic acid-based assays can be employed to examine the microbes in a sample. For example, some nucleic acid-based population methods use denaturation and reannealing kinetics to derive an indirect estimate of the percent (%) guanine and cytosine nucleotides (G+C) content of the DNA in the sample. This method has been used to characterize a bacterial community in the ileum and cecum of the GIT in poultry, and to examine how diet and other variables modulate the microbial communities in the GITs of animals (Apajalahti et al., 2001, Appt Environ. Microbiol., 67:5656-67). The % G+C technique provides an overall view of the microbial community and is sensitive only to massive changes in the make-up of the community.

Genetic fingerprinting of a sample is another method that can be used to examine a sample for the presence of microbes. Genetic fingerprinting utilizes random-sequence oligonucleotide primers that hybridize with sequence-specificity to random sequences throughout the genome. Amplification results in a multitude of products. The distribution of amplification products is referred to as a genetic fingerprint. Particular patterns can be associated with microbes in the sample. Genetic fingerprinting, however, lacks the ability to conclusively identify specific microbial species.

Denaturing or temperature gradient gel electrophoresis (DGGE or TGGE) is another technique that can be used to examine a sample for the presence of microbes. As amplification products are electrophoresed in gradients with increasing denaturant or temperature, the double-stranded molecule melts and its mobility is reduced. The melting behavior is determined by the nucleotide sequence, and unique sequences will resolve into individual bands. Thus, a D/TGGE gel yields a genetic fingerprint characteristic of the microbial community, and the relative intensity of each band reflects the abundance of the corresponding microorganism. An alternative format includes single-stranded conformation polymorphism (SSCP). SSCP relies on the same physical basis as %G+C renaturation methods, but reflects a significant improvement over such methods.

In addition, the microbes in a sample can be evaluated using terminal restriction fragment length polymorphism (TRFLP). Amplification products can be analyzed for the presence of known sequence motifs using restriction endonucleases that recognize and cleave double-stranded nucleic acids at these motifs. For example, the enzyme Hhal cuts at 5′-GCGC-3′ sites. Using a fluorescently-labeled primer to tag one end of the amplification product and Hhal to digest the products, resolution of this mixture by electrophoresis will yield a series of fluorescent bands whose lengths are determined by how far a 5′-GCGC-3′ motif lies from the terminal tag. TRFLP profiles can be generated using a variety of restriction enzymes, and can be correlated with changes in the microbial population. For example, a TRFLP database for 16S rRNA sequences has been set up at Michigan State University to allow researchers to design experimental parameters (e.g., choice of enzyme and primer combinations). The principal advantages of TRFLP are its robustness and its low cost. Unlike D/TGGE, experimental conditions need not-be stringently controlled since the profiles are size-based and thus can be generated by a variety of gel systems, including automated DNA sequencing machines. Alternative approaches include “amplified ribosomal DNA restriction analysis (AADRA)” in which the entire amplification product, rather than just the terminal fragment, is considered. AADRA, however, becomes unmanageable with communities containing many species.

Genotyping of 16S ribosomal DNA (rDNA) is another way to examine the microbes in a sample. 16S rDNA sequences are universal, composed both of highly conserved regions, which allows for the design of common amplification primers, and open reading frame (ORF) regions with sequence variation, which allows for phylogenetic differentiation. 16S ribosomal sequences are relatively abundant in the RNA form. In addition to amplification using oligonucleotide primers, genotyping of 16S rDNA can be performed using other methods including restriction fragment length polymorphism (RFLP) with Southern blotting.

Other targets for genotyping include genes encoding components of RNA polymerase, translation elongation factors, gyrase, and chaperoning. Such protein-encoding sequences may evolve more rapidly than those encoding structural RNAs. Thus, the sequences of protein-encoding sequences in closely related species may have diverged more in closely related species and may provide more discriminatory information. The choice of which target sequence to use depends on whether the sequences provide both broad coverage and discriminatory power. Ideally, the target should be present in all members of a given microbial community, be amplified from each member with equal efficiency using common primers, yet have distinct sequences. Multiple targets may in fact prove necessary for particular applications.

Chaperonin 60 (cpn60) nucleic acid sequences are particularly useful targets for genotyping and can be used to examine the microbes in a sample from grain or a related product. Chaperonin proteins are molecular chaperones required for proper folding of polypeptides in vivo. cpn60 is found universally in prokaryotes and in the organelles of eukaryotes, and can be used as a species-specific target and/or probe for identification and classification of microorganisms. Sequence diversity within this protein-encoding gene appears greater between and within bacterial genera than for 16S rDNA sequences, thus making cpn60 a superior target sequence having more distinguishing power for microbial identification at the species level than 16S rDNA sequences.

PCR oligonucleotide primers that universally amplify a 552-558 base pair (bp) segment of cpn60 from numerous microorganisms have been generated (see, for example, U.S. Pat. Nos. 5,708,160 and 5,989,821), and the nucleotide sequence of this region of cpn60 has been evaluated as a tool for microbial analysis. The utility of the sequence diversity in cpn60 has been demonstrated, in part, by cross hybridization experiments using nylon membranes spotted with cpn60 amplification products from typed strains probed with labeled amplification product from unknown isolates. By manipulating stringency conditions, hybridization can be limited to targets having >75% identity (e.g., >80%, >85%, >90%, >95% identify) to the unknown isolate. This level of cross hybridization allows for clear differentiation of species within genera.

Nucleic acid hybridization is another method that can be used to examine the microbes contaminating grain and related products. Probing amplification products with species-specific hybridization probes is one of the most powerful analytical tools available for profiling. The physical matrix for hybridization can be nylon membranes (e.g., macroarrays) or microarrays (e.g., microchips), incorporation of the hybridization probes into the amplification reaction (e.g., TaqMan or Molecular Beacon technology), solution-based methods (e.g., ORIGEN technology), or any one of numerous approaches devised for clinical diagnostics. Probes can be designed to preferentially hybridize to amplification products from individual species or to discriminate species phylogenetically.

The microbes in a sample also can be examined by cloning and sequencing microbial nucleic acids present in the sample. Cloning of individual nucleic acids into Escherichia coli and sequencing each nucleic acid gives the highest density of information but requires the most effort. Although sequencing nucleic acids is automated, routine monitoring of changes in the microbial profile of an animal by cloning and sequencing nucleic acids from the microorganisms still requires considerable time and effort.

Representative Nucleic-acid Based Assays

PCR Assays

Nucleic acid-based methods for identifying and/or quantitating the amount of a microbe in a sample can include amplification of a nucleic acid. Amplification methods such as PCR provide powerful means by which to increase the amount of a particular nucleic acid sequence. Nucleic acid hybridization also can be included in determining the presence or absence of a microbe in a sample. Probing amplification products with species-specific hybridization probes is one of the most powerful analytical tools available for profiling. The physical matrix for hybridization can be a nylon membrane (e.g., a macroarray) or a microarray (e.g., a microchip), incorporation of one or more hybridization probes into an amplification reaction (e.g., TaqMan® or Molecular Beacon technology), solution-based methods (e.g., ORIGEN technology), or any one of numerous approaches devised for clinical diagnostics. As discussed above, probes can be designed to preferentially hybridize to amplification products from individual species or to discriminate specific species.

U.S. Pat. Nos. 4,683,202, 4,683,195, 4,800,159, and 4,965,188 disclose conventional PCR techniques. PCR typically employs two oligonucleotide primers that bind to a selected nucleic acid template (e.g., DNA or RNA). Primers useful in the present invention include oligonucleotide primers capable of acting as a point of initiation of nucleic acid synthesis within or adjacent to sequences. A primer can be purified from a restriction digest by conventional methods, or can be produced synthetically. Primers typically are single-stranded for maximum efficiency in amplification, but a primer can be double-stranded. Double-stranded primers are first denatured (e.g., treated with heat) to separate the strands before use in amplification. Primers can be designed to amplify a nucleotide sequence from a particular microbial species, or can be designed to amplify a sequence from more than one species. Primers that can be used to amplify a nucleotide sequence from more than one species are referred to herein as “universal primers.”

PCR assays can employ template nucleic acids such as DNA or RNA, including messenger RNA (mRNA). The template nucleic acid need not be purified; it can be a minor fraction of a complex mixture, such as a microbial nucleic acid contained in animal cells. Template DNA or RNA can be extracted from a biological or non-biological sample using routine techniques such as those described in Diagnostic Molecular Microbiology: Principles and Applications (Persing et al. (eds.), 1993, American Society for Microbiology, Washington D.C.). Nucleic acids can be obtained from any of a number of sources, including plasmids, bacteria, yeast, organelles, and higher organisms such as plants and animals. Standard conditions for generating a PCR product are well known in the art (see, e.g., PCR Primer: A Laboratory Manual, Dieffenbach and Dveksler (eds.), Cold Spring Harbor Laboratory Press, 1995).

Once a PCR amplification product is generated, it can be detected by, for example, electrophoresis and/or hybridization. One type of hybridization commonly used is a Southern blot. Southern blot hybridization between nucleic acid molecules is discussed in detail in Sambrook et al. (1989, Molecular Cloning: A Laboratory Manual, 2^{nd} Ed., Cold Spring Harbor Laboratory Press, Cold Spring Harbor, N.Y.; Sections 7.37-7.57, 9.47-9.57, 11.7-11.8, and 11.45-11.57).

If microbial contamination is present in a sample, a hybridization complex is produced between the microbial nucleic acid and an oligonucleotide probe. For oligonucleotide probes less than about 100 nucleotides, Sambrook et al. discloses suitable Southern blot conditions in Sections 11.45-11.46. The Tm between a sequence that is less than 100 nucleotides in length and a second sequence can be calculated using the formula provided in Section 11.46. Sambrook et al. additionally discloses prehybridization and hybridization conditions for a Southern blot that uses oligonucleotide probes greater than about 100 nucleotides (see Sections 9.47-9.52). Hybridizations with an oligonucleotide greater than 100 nucleotides generally are performed 15-25° C. below the Tm. The Tm between a sequence greater than 100 nucleotides in length and a second sequence can be calculated using the formula provided in Sections 9.50-9.51 of Sambrook et al. Additionally, Sambrook et al. recommends the conditions indicated in Section 9.54 for washing a Southern blot that has been probed with an oligonucleotide greater than about 100 nucleotides.

The conditions under which membranes containing nucleic acids are prehybridized and hybridized, as well as the conditions under which membranes containing nucleic acids are washed to remove excess and non-specifically bound probe can play a significant role in the stringency of the hybridization. Such hybridizations can be performed, where appropriate, under moderate or high stringency conditions. Such conditions are described, for example, in Sambrook et al. section 11.45-11.46. For example, washing conditions can be made more stringent by decreasing the salt concentration in the wash solutions and/or by increasing the temperature at which the washes are performed. In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium.

It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to nucleic acids from a microorganism (e.g., a contaminating microbe) but not to control nucleic acids if hybridization to nucleic acid from a microorganism is at least 5-fold (e.g., at least 6-fold, 7-fold, 8-fold, 9-fold, 10-fold, 20-fold, 50-fold, or 100-fold) greater than hybridization to control nucleic acid. The amount of hybridization can be quantitated directly on a membrane or from an autoradiograph using, for example, a Phosphorlmager or a Densitometer (Molecular Dynamics, Sunnyvale, Calif.).

Another form of hybridization involves the use of FRET technology. FRET technology (see, for example, U.S. Pat. Nos. 4,996,143, 5,565,322, 5,849,489, and 6,162,603) is based on the concept that when a donor fluorescent moiety and a corresponding acceptor fluorescent moiety are positioned within a certain distance of each other, energy transfer taking place between the two fluorescent moieties can be visualized or otherwise detected and quantitated. One or two oligonucleotide probes containing fluorescent moieties, can hybridize to an amplification product at particular positions determined by the complementarity of the oligonucleotide probe(s) to the target nucleic acid sequence. Generally, and depending on the particular strategy for detection (e.g., Molecular Beacon technology) hybridization of the oligonucleotide probe(s) to the amplification product at the appropriate positions generates a FRET signal. Detection of FRET can occur in real-time, such that the increase in an amplification product after each cycle of a PCR assay is detected and, in some embodiments, quantified.

Fluorescent analysis and quantification can be carried out using, for example, a photon counting epifluorescent microscope system (containing the appropriate dichroic mirror and filters for monitoring fluorescent emission in a particular range of wavelengths), a photon counting photomultiplier system, or a fluorometer. Excitation to initiate energy transfer can be carried out with an argon ion laser, a high intensity mercury arc lamp, a fiber optic light source, or another high intensity light source appropriately filtered for excitation in the desired range.

Fluorescent moieties can be, for example, a donor moiety and a corresponding acceptor moiety. As used herein with respect to donor and corresponding acceptor fluorescent moieties, “corresponding” refers to an acceptor fluorescent moiety having an emission spectrum that overlaps the excitation spectrum of the donor fluorescent moiety. The wavelength maximum of the emission spectrum of an acceptor fluorescent moiety typically should be at least 100 nm greater than the wavelength maximum of the excitation spectrum of the donor fluorescent moiety, such that efficient non-radiative energy transfer can be produced therebetween.

Fluorescent donor and corresponding acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen with an excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC™-Red 640, LC™-Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, and other chelates of Lanthanide ions (e.g., Europium, or Terbium). Donor and acceptor fluorescent moieties can be obtained from, for example, Molecular Probes, Inc. (Eugene, Oreg.) or Sigma Chemical Co. (St. Louis, Mo.).

Donor and acceptor fluorescent moieties can be attached to probe oligonucleotides via linker arms. The length of each linker arm is important, as the linker arms will affect the distance between the donor and acceptor fluorescent moieties. The length of a linker arm for the purpose of the present invention is the distance in Angstroms (Å) from the nucleotide base to the fluorescent moiety. In general, a linker arm is from about 10 to about 25 Å in length. The linker arm may be of the kind described in WO 84/03285, for example. WO 84/03285 also discloses methods for attaching linker arms to a particular nucleotide base, as well as methods for attaching fluorescent moieties to a linker arm.

The amount of FRET corresponds to the amount of amplification product, which in turn corresponds to the amount of template nucleic acid present in the sample. Similarly, the amount of template nucleic acid corresponds to the amount of microbial organism present in the sample. Therefore, the amount of FRET produced when amplifying nucleic acid obtained from a biological sample can be correlated to the amount of a microorganism. Typically, the amount of a microorganism in a sample can be quantified by comparing to the amount of FRET produced from amplified nucleic acid obtained from known amounts of the microorganism (e.g., a standard curve). Accurate quantitation requires measuring the amount of FRET while amplification is increasing linearly. In addition, there must be an excess of probe in the reaction. Furthermore, the amount of FRET produced in the known samples used for comparison purposes can be standardized for particular reaction conditions, such that it is not necessary to isolate and amplify samples from every microorganism for comparison purposes.

As an alternative to FRET, an amplification product can be detected using, for example, a fluorescent DNA binding dye (e.g., SYBRGreen® or SYBRGold® (Molecular Probes)). Upon interaction with an amplification product, such DNA binding dyes emit a fluorescent signal after excitation with light at a suitable wavelength. A double-stranded DNA binding dye such as a nucleic acid intercalating dye also can be used. When double-stranded DNA binding dyes are used, a melting curve analysis usually is performed for confirmation of the presence of the amplification product.

Melting curve analysis is an additional step that can be included in a cycling profile. Melting curve analysis is based on the fact that a nucleic acid sequence melts at a characteristic temperature (Tm), which is defined as the temperature at which half of the DNA duplexes have separated into single strands. The melting temperature of a DNA molecule depends primarily upon its nucleotide composition. A DNA molecule rich in G and C nucleotides has a higher Tm than one having an abundance of A and T nucleotides. The temperature at which the FRET signal is lost correlates with the melting temperature of a probe from an amplification product. Similarly, the temperature at which signal is generated correlates with the annealing temperature of a probe with an amplification product. The melting temperature(s) of probes from an amplification product can confirm the presence or absence of containing such sequences in a sample, and can be used to quantify the amount of a particular species. For example, a universal probe that hybridizes to a variable region within a sequence will have a Tm that depends upon the sequence to which it hybridizes. By observing a temperature-dependent, step-wise decrease in fluorescence of a sample as it is heated, the particular species in the sample can be identified and the relative amounts of the species in the sample can be determined.

Within each thermocycler run, control samples can be cycled as well. Positive control samples can amplify a nucleic acid control template (e.g., a nucleic acid other than the target nucleic acid) using, for example, control primers and control probes. Positive control samples also can amplify, for example, a plasmid construct containing a target nucleic acid molecule. Such a plasmid control can be amplified internally (e.g., within the sample) or in a separate sample run side-by-side with the test samples. Each thermocycler run also should include a negative control that, for example, lacks template DNA. Such controls are indicators of the success or failure of the amplification, hybridization and/or FRET reaction. Therefore, control reactions can readily determine, for example, the ability of primers to anneal with sequence-specificity and to initiate elongation, as well as the ability of probes to hybridize with sequence-specificity and for FRET to occur.

In one embodiment, methods of the invention include steps to avoid contamination. For example, an enzymatic method utilizing uracil-DNA glycosylase is described in U.S. Pat. Nos. 5,035,996, 5,683,896 and 5,945,313, and can be used to reduce or eliminate contamination between one thermocycler run and the next. In addition, standard laboratory containment practices and procedures are desirable when performing methods of the invention. Containment practices and procedures include, but are not limited to, separate work areas for different steps of a method, containment hoods, barrier filter pipette tips and dedicated air displacement pipettes. Consistent containment practices and procedures by personnel are necessary for accuracy in a diagnostic laboratory handling clinical samples.

It is understood that the present invention is not limited by the configuration of one or more commercially available instruments.

Fluorescent In Situ Hybridization (FISH)

In situ hybridization methods such as FISH also can be used to determine a microbial profile. In general, in situ hybridization methods provided herein include the steps of fixing a biological sample, hybridizing a probe to target DNA contained within the fixed biological sample, washing to remove non-specific binding, detecting the hybridized probe, and quantifying the amount of hybridized probe.

Typically, cells are harvested from a biological sample using standard techniques. For example, cells can be harvested by centrifuging a biological sample and resuspending the pelleted cells in, for example, phosphate-buffered saline (PBS). After re-centrifuging the cell suspension to obtain a cell pellet, the cells can be fixed in a solution such as an acid alcohol solution, an acid acetone solution, or an aldehyde such as formaldehyde, paraformaldehyde, or glutaraldehyde. For example, a fixative containing methanol and glacial acetic acid in a 3:1 ratio, respectively, can be used as a fixative. A neutral buffered formalin solution also can be used (e.g., a solution containing approximately 1% to 10% of 37-40% formaldehyde in an aqueous solution of sodium phosphate). Slides containing the cells can be prepared by removing a majority of the fixative, leaving the concentrated cells suspended in only a portion of the solution.

The cell suspension is applied to slides such that the cells do not overlap on the slide. Cell density can be measured by a light or phase contrast microscope. For example, cells harvested from a 20 to 100 ml urine sample typically are resuspended in a final volume of about 100 to 200 μl of fixative. Three volumes of this suspension (e.g., 3, 10, and 30 μl), are then dropped into 6 mm wells of a slide. The cellularity (i.e., the density of cells) in these wells is then assessed with a phase contrast microscope. If the well containing the greatest volume of cell suspension does not have enough cells, the cell suspension can be concentrated and placed in another well.

Probes for FISH are chosen for maximal sensitivity and specificity. Using a set of probes (e.g., two or more) can provide greater sensitivity and specificity than the use of any one probe. Probes typically are about 50 to about 2×10³ nucleotides in length (e.g., 50, 75, 100, 200, 300, 400, 500, 750, 1000, 1500, or 2000 nucleotides in length). Longer probes can comprise smaller fragments of about 100 to about 500 nucleotides in length. Probes that hybridize with locus-specific DNA can be obtained commercially from, for example, Vysis, Inc. (Downers Grove, Ill.), Molecular Probes, Inc. (Eugene, Oreg.), or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, and chromosomes purified by flow cytometry or microdissection. The region of interest can be isolated through cloning, or by site-specific amplification via PCR. See, for example, Nath and Johnson, Biotechnic Histochem., 1998, 73(1):6-22, Wheeless et al., Cytometry, 1994, 17:319-326, and U.S. Pat. No. 5,491,224.

Probes for FISH typically are directly labeled with a fluorescent moiety (also referred to as a fluorophore), an organic molecule that fluoresces after absorbing light of lower wavelength/higher energy. The fluorescent moiety allows the probe to be visualized without a secondary detection molecule. After covalently attaching a fluorophore to a nucleotide, the nucleotide can be directly incorporated into a probe using standard techniques such as nick translation, random priming, and PCR labeling. Alternatively, deoxycytidine nucleotides within a probe can be transaminated with a linker. A fluorophore then can be covalently attached to the transaminated deoxycytidine nucleotides. See U.S. Pat. No. 5,491,224. The amount of fluorophore incorporated into a probe can be known or determined, and this value in turn can be used to determine the amount of nucleic acid to which the probe binds. In conjunction with analysis of samples (e.g., a serial dilution of a sample) containing known numbers of microbial organisms, the number of microbial organisms in a biological or non-biological sample can be determined.

When more than one probe is used, fluorescent moieties of different colors can be chosen such that each probe in the set can be distinctly visualized and quantitated. For example, a combination of the following fluorophores may be used: 7-amino-4-methylcoumarin-3-acetic acid (AMCA), Texas Red™ (Molecular Probes, Inc.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and Cascade™ blue acetylazide (Molecular Probes, Inc.). Probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine and quantitate the hybridization pattern of the probes.

Probes also can be indirectly labeled with biotin or digoxygenin, or labeled with radioactive isotopes such as ³²P and ³H, although secondary detection molecules or further processing then may be required to visualize the probes and quantify the amount of hybridization. For example, a probe indirectly labeled with biotin can be detected and quantitated using avidin conjugated to a detectable enzymatic marker such as alkaline phosphatase or horseradish peroxidase. Enzymatic markers can be detected and quantitated in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Prior to in situ hybridization, the probes and the chromosomal DNA contained within the cell each are denatured. Denaturation typically is performed by incubating the nucleic acids in the presence of high pH, heat (e.g., temperatures from about 70° C. to about 95° C.), organic solvents such as formamide and tetraalkylammonium halides, or combinations thereof. For example, chromosomal DNA can be denatured by a combination of temperatures above 70° C. (e.g., about 73° C.) and a denaturation buffer containing 70% formamide and 2×SSC (0.3 M sodium chloride and 0.03 M sodium citrate). Denaturation conditions typically are established such that cell morphology is preserved. Probes can be denatured by heat (e.g., by heating to about 73° C. for about five minutes).

After removal of denaturing chemicals or conditions, probes are annealed to the chromosomal DNA under hybridizing conditions. “Hybridizing conditions” are conditions that facilitate annealing between a probe and target chromosomal DNA. Hybridization conditions vary, depending on the concentrations, base compositions, complexities, and lengths of the probes, as well as salt concentrations, temperatures, and length of incubation. The higher the concentration of probe, the higher the probability is of forming a hybrid. For example, in situ hybridizations typically are performed in hybridization buffer containing 1-2×SSC, 50% formamide, and blocking DNA to suppress non-specific hybridization. In general, hybridization conditions, as described above, include temperatures of about 25° C. to about 55° C., and incubation times of about 0.5 hours to about 96 hours. More particularly, hybridization can be performed at about 32° C. to about 40° C. for about 2 to about 16 hours.

Non-specific binding of probes to DNA outside of the target region can be removed by a series of washes. The temperature and concentration of salt in each wash depend on the desired stringency. For example, for high stringency conditions, washes can be carried out at about 65° C. to about 80° C., using 0.2× to about 2×SSC, and about 0.1% to about 1% of a non-ionic detergent such as Nonidet P-40 (NP40). Stringency can be lowered by decreasing the temperature of the washes or by increasing the concentration of salt in the washes.

mRNA-based Assays

Alternatively, in order to test for the presence or absence of, or measure the level of, a specific mRNA in a sample, e.g., a sample comprising cells, the cells can be lysed and total RNA can be purified or semi-purified from lysates by any of a variety of methods known in the art. Methods of detecting or measuring levels of particular mRNA transcripts are also familiar to those in the art. Such assays include, without limitation, hybridization assays using detectably labeled specific nucleic acid (DNA or RNA) probes and quantitative or semi-quantitative RT-PCR methodologies employing appropriate oligonucleotide primers. Additional methods for quantitating mRNA in cell lysates include RNA protection assays and serial analysis of gene expression (SAGE). Alternatively, qualitative, quantitative, or semi-quantitative in situ hybridization assays can be carried out using, for example, samples such as tissue sections or unlysed cell suspensions, and detectably (e.g., fluorescently, isotopically, or enzymatically) labeled DNA or RNA probes.

Representative Polypeptide-based Assays

The invention also features polypeptide-based assays. A microbial polypeptide can be used as a universal target to determine the presence or absence of one or more microbes, and further used as species-specific targets and/or probes for the identification and classification of specific microbes. Such assays can be used on their own or in conjunction with other procedures (e.g., nucleic acid-based assays) to detect and monitor grain and related products.

In the assays of the invention, the presence or absence of a microbial polypeptide is detected and/or its level is measured.

Methods of detecting or measuring the levels of a protein of interest (e.g., a cpn60 protein, or cpn60-specific polypeptides) in cells are known in the art. Many such methods employ antibodies (e.g., polyclonal antibodies or mAbs) that bind specifically to the protein.

Antibodies and Antibody-based Assays

Antibodies having specific binding affinities for a microbial polypeptide may be produced through standard methods. Such antibodies may be referred to as anti-microbial antibodies. As used herein, the terms “antibody” or “antibodies” include intact molecules as well as fragments thereof which are capable of binding to an epitopic determinant of a specific polypeptide. The term “epitope” refers to an antigenic determinant on an antigen to which the paratope of an antibody binds. Epitopic determinants usually consist of chemically active surface groupings of molecules such as amino acids or sugar side chains, and typically have specific three-dimensional structural characteristics, as well as specific charge characteristics. Epitopes generally have at least five contiguous amino acids (a continuous epitope), or alternatively can be a set of noncontiguous amino acids that define a particular structure (e.g., a conformational epitope). The terms “antibody” and “antibodies” include polyclonal antibodies, monoclonal antibodies, humanized or chimeric antibodies, single chain Fv antibody fragments, Fab fragments, and F(ab)₂ fragments.

Antibodies may be specific for a particular polypeptide specific to a genus or species of microbe. Alternatively, they may be cross-reactive with polypeptides common to more than one genera or species. For example, such antibodies may bind to common epitopes present in homologs from different microbes. As used herein, such antibodies with specificity for a polypeptide from more than one microbe are termed “universal” antibodies. For example, certain antibodies may bind to common epitopes present in homologs from a number of different microbes. Certain of such antibodies thus may be termed able to detect the presence or absence of any of a large number of microbes in a sample.

In certain embodiments of the method described herein, depending on the grain or related product and the purpose for monitoring, it may be sufficient to determine simply whether or not any microbe is present, and optionally the relative concentration or amount of the microbe. Such detection may occur through, e.g., the use of one or more “universal” antibodies.

In other embodiments, the identification of the particular microbe may be preferred. Accordingly, an antibody having specific binding affinity for a polypeptide from a particular organism may be employed, either alone or in conjunction with a universal antibody; such antibodies are referred to as “specific” antibodies herein. The universal and specific antibodies may be employed simultaneously or in series. For example, a universal antibody may be used as a first screen to determine the presence or absence of a polypeptide. Subsequently, a specific antibody, such as one specific for a polypeptide from a particular microbe, e.g., Campylobacter jejuni, may be employed. In such assays, monoclonal antibodies may be particularly useful (e.g., sensitive) to identify polypeptides of a particular microbe.

In general, a protein of interest (e.g., a protein against which one wishes to prepare antibodies) is produced recombinantly, by chemical synthesis, or by purification of the native protein, and then used to immunize animals. As used herein, an intact protein or a polypeptide fragment thereof may be employed, provided that the polypeptide is capable of generating the desired immune response. See, for example, WO 200265129 for examples of epitopic sequences that bind to human antibodies against Chlamydia trachomatis; such epitopic sequences may be useful in generating antibodies against Chlamydia spp. for use in the present invention. See also U.S. Pat. No. 6,497,880, which sets forth nucleic acid sequences, amino acid sequences, expression vectors, purified proteins, antibodies, etc. specific to Aspergillus fumigatus and Candida glabrata. Purified Aspergillus fumigatus and Candida glabrata cpn60 proteins, for example, or proteolytically or synthetically generated fragments thereof, can be used to immunize animals to generate antibodies for use in the methods of the present invention. Finally, see WO 02/57784, disclosing substantially purified Chlamydia hsp60 (cpn60) polypeptides. Such polypeptides may also be used to generate antibodies for use in the methods of the present invention.

As discussed previously, one may wish to prepare universal or specific antibodies to a protein or polypeptide. A polypeptide may be used to generate a universal antibody, for example, if it maintains an epitope that is common to at least two proteins, or, e.g., to all proteins from each species that one wishes to detect. Alternatively, a protein or polypeptide may be used to generate antibodies specific for a particular protein or polypeptide present in a specific microbe, e.g., only Campylobacter jejuni.

Various host animals including, for example, rabbits, chickens, mice, guinea pigs, and rats, can be immunized by injection of the protein of interest. Adjuvants can be used to increase the immunological response depending on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin (KLH), and dinitrophenol. Polyclonal antibodies are heterogeneous populations of antibody molecules that are specific for a particular antigen, which are contained in the sera of the immunized animals. Monoclonal antibodies, which are homogeneous populations of antibodies to a particular epitope contained within an antigen, can be prepared using standard hybridoma technology. In particular, monoclonal antibodies can be obtained by any technique that provides for the production of antibody molecules by continuous cell lines in culture such as described by Kohler, G. et al., Nature, 1975, 256:495, the human B-cell hybridoma technique (Kosbor et al., Immunology Today, 1983, 4:72; Cole et al., Proc. Natl. Acad. Sci. USA, 1983, 80:2026), and the EBV-hybridoma technique (Cole et al., “Monoclonal Antibodies and Cancer Therapy”, Alan R. Liss, Inc., 1983, pp. 77-96). Such antibodies can be of any immunoglobulin class including IgG, IgM, IgE, IgA, IgD, and any subclass thereof. The hybridoma producing the monoclonal antibodies of the invention can be cultivated in vitro or in vivo.

A chimeric antibody is a molecule in which different portions are derived from different animal species, such as those having a variable region derived from a murine monoclonal antibody and a human immunoglobulin constant region. Chimeric antibodies can be produced using standard techniques.

Antibody fragments that have specific binding affinity for a polypeptide can be generated by known techniques. For example, such fragments include, but are not limited to, F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed. See, for example, Huse et al., 1989, Science, 246:1275. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques. See, for example, U.S. Pat. No. 4,946,778.

Once produced, antibodies or fragments thereof are tested for recognition of the protein or polypeptide by standard immunoassay methods including, for example, ELISA techniques, countercurrent immuno-electrophoresis (CIEP), radioimmunassays (RIA), radioimmunoprecipitations, dot blots, inhibition or competition assays, sandwich assays, immunostick (dipstick) assays, immunochromatographic assays, immunofiltration assays, latex beat agglutination assays, immunofluorescent assays, biosensor assays. See, Short Protocols in Molecular Biology, Chapter 11, Green Publishing Associates and John Wiley & Sons, Edited by Ausubel, F. M et al., 1992; Antibodies: A Laboratory Manual, Harlow and Lane (eds.), Cold Spring Harbor Laboratory Press, 1988; and U.S. Pat. Nos. 4,376,110; 4,486,530; and 6,497,880. Antibodies or fragments can also be tested for their ability to react universally, e.g., with proteins or polypeptides from more than one genus or species of microbe or specifically with a particular protein from a specific organism.

In antibody assays, the antibody itself or a secondary antibody that binds to it can be detectably labeled. Alternatively, the antibody can be conjugated with biotin, and detectably labeled avidin (a protein that binds to biotin) can be used to detect the presence of the biotinylated antibody. Combinations of these approaches (including “multi-layer” assays) familiar to those in the art can be used to enhance the sensitivity of assays. Some of these assays (e.g., immunohistological methods or fluorescence flow cytometry) can be applied to histological sections or unlysed cell suspensions. The methods described below for detecting a polypeptide in a liquid sample can also be used to detect a polypeptide in cell lysates.

Methods of detecting a polypeptide in a liquid sample generally involve contacting a sample of interest with an antibody that binds to the polypeptide and testing for binding of the antibody to a component of the sample. In such assays, the antibody need not be detectably labeled and can be used without a second antibody. For example, an antibody having binding affinity for a polypeptide may be bound to an appropriate solid substrate and then exposed to the sample. Binding of a polypeptide to the antibody on the solid substrate may be detected by exploiting the phenomenon of surface plasmon resonance, which results in a change in the intensity of surface plasmon resonance upon binding that can be detected qualitatively or quantitatively by an appropriate instrument, e.g., a Biacore apparatus (Biacore International AB, Rapsgatan, Sweden).

Moreover, assays for detection of a polypeptide in a liquid sample can involve the use, for example, of: (a) a single antibody specific for a polypeptide that is detectably labeled; (b) an unlabeled antibody that is specific for a polypeptide and a detectably labeled secondary antibody; or (c) a biotinylated antibody specific for a polypeptide and detectably labeled avidin. In addition, combinations of these approaches (including “multi-layer” assays) familiar to those in the art can be used to enhance the sensitivity of assays. In these assays, the sample or an aliquot of the sample suspected of containing a microbe can be immobilized on a solid substrate, such as a nylon or nitrocellulose membrane, by, for example, “spotting” an aliquot of the liquid sample or by blotting of an electrophoretic gel on which the sample or an aliquot of the sample has been subjected to electrophoretic separation. The presence or amount of polypeptide on the solid substrate is then assayed using any of the above-described forms of the polypeptide specific antibody and, where required, appropriate detectably labeled secondary antibodies or avidin.

The invention also features “sandwich” assays. In these sandwich assays, instead of immobilizing samples on solid substrates by the methods described above, any polypeptide that may be present in a sample can be immobilized on the solid substrate by, prior to exposing the solid substrate to the sample, conjugating a second (“capture”) antibody (polyclonal or mAb) specific for the polypeptide to the solid substrate by any of a variety of methods known in the art. In exposing the sample to the solid substrate with the second antibody specific for the polypeptide bound to it, any polypeptide in the sample (or sample aliquot) will bind to the second antibody on the solid substrate. The presence or amount of polypeptide bound to the conjugated second antibody is then assayed using a “detection” antibody specific for a polypeptide by methods essentially the same as those described above using a single antibody specific for a polypeptide. It is understood that in these sandwich assays, the capture antibody should not bind to the same epitope (or range of epitopes in the case of a polyclonal antibody) as the detection antibody. Thus, if a mAb is used as a capture antibody, the detection antibody can be either: (a) another mAb that binds to an epitope that is either completely physically separated from or only partially overlaps with the epitope to which the capture mAb binds; or (b) a polyclonal antibody that binds to epitopes other than or in addition to that to which the capture mAb binds. On the other hand, if a polyclonal antibody is used as a capture antibody, the detection antibody can be either (a) a mAb that binds to an epitope to that is either completely physically separated from or partially overlaps with any of the epitopes to which the capture polyclonal antibody binds; or (b) a polyclonal antibody that binds to epitopes other than or in addition to that to which the capture polyclonal antibody binds. Assays that involve the used of a capture and detection antibody include sandwich ELISA assays, sandwich Western blotting assays, and sandwich immunomagnetic detection assays.

Suitable solid substrates to which the capture antibody can be bound include, without limitation, the plastic bottoms and sides of wells of microtiter plates, membranes such as nylon or nitrocellulose membranes, and polymeric (e.g., without limitation, agarose, cellulose, or polyacrylamide) beads or particles. It is noted that antibodies bound to such beads or particles can also be used for immunoaffinity purification of polypeptides. Dipstick/immunostick formats can employ a solid phase, e.g., polystyrene, paddle or dispstick.

Methods of detecting or for quantifying a detectable label depend on the nature of the label and are known in the art. Appropriate labels include, without limitation, radionuclides (e.g., ¹²⁵I, ¹³¹I, ³⁵S, ³H, ³²P, ³³P, or ¹⁴C), fluorescent moieties (e.g., fluorescein, rhodamine, or phycoerythrin), luminescent moieties (e.g., Qdo™ nanoparticles supplied by the Quantum Dot Corporation, Palo Alto, Calif.), compounds that absorb light of a defined wavelength, or enzymes (e.g., alkaline phosphatase or horseradish peroxidase). The products of reactions catalyzed by appropriate enzymes can be, without limitation, fluorescent, luminescent, or radioactive, or they may absorb visible or ultraviolet light. Examples of detectors include, without limitation, x-ray film, radioactivity counters, scintillation counters, spectrophotometers, calorimeters, fluorometers, luminometers, and densitometers.

The methods of the present invention may employ a control sample. In assays to detect the presence or absence of a microbe, the concentration of a microbial polypeptide may be compared to a control sample. The control sample may be taken from the same source of grain or related product, e.g., in a different location known to be uncontaminated, or can be a control sample taken from uncontaminated grain or related product. Alternatively, the control sample may be taken from the same source of grain or related product but at an earlier or later time-point when the source was known to be uncontaminated. A significantly higher concentration of polypeptide in the suspect sample relative to the control sample would indicate the presence of a microbe.

It is understood that, while the above descriptions of the diagnostic assays may refer to assays on particular samples, the assays can also be carried out on any of the other fluid or solubilized samples listed herein, such as water samples or buffer samples (e.g., buffer used to extract a sample from a swab).

Other polypeptide-based Detection Assays

The present invention also contemplates the use of other analytical techniques for detecting microbial polypeptides. Recent analytical instrumentation and methodology advances that have arisen in the context of proteomics research are applicable in the methods of the present invention. See, generally, Jungblut, Microbes & Infection 3 (2001): 831-840; MacBeath & Schreiber, Science 289 (2000): 1760-1763; Madoz-Gdrpide et al., Proteomics 1 (2001): 1279-1287; Patterson, Physiological Genomics 2 (2000): 59-65; and Schevchenko et al., Analytical Chemistry 72 (2000):2132-2141. Mass-spectrophotometric techniques have been increasingly used to detect and identify proteins and protein fragments at low levels, e.g., fmol or pmol. Mass spectrometry has become a major analytical tool for protein and proteomics research because of advancements in the instrumentation used for biomolecular ionization, electrospray ionization (ESI), and matrix-assisted laser desorption-ionization (MALDI). MALDI is usually combined with a time-of-flight (TOF) mass analyzer. Typically, 0.5 μl of sample that contains 1-10 pmol of protein or peptide is mixed with an equal volume of a saturated matrix solution and allowed to dry, resulting in the co-crystallization of the analyte with the matrix. Matrix compounds that are used include sinapic acid and α-hydroxycinnamic acid. The co-crystallized material on the target plate is irradiated with a nitrogen laser pulse, e.g., at a wavelength of 337 nm, to volatilize and ionize the protein or peptide molecules. A strong acceleration field is switched on, and the ionized molecules move down the flight tube to a detector. The amount of time required to reach the detector is related to the mass-to-charge ratio. Proteolytic mass mapping and tandem mass spectrometry, when combined with searches of protein and protein fragment databases, can also be employed to detect and identify polypeptides. See, for example, Downard, J. Mass. Spectrom. 35:493-503 (2000).

Biomolecular interaction analysis mass spectrometry (BIA-MS) is another suitable technique for detecting interactions between polypeptides and antibodies. This technology detects molecules bound to a ligand that is covalently attached to a surface. As the density of biomaterial on the surface increases, changes occur in the refractive index at the solution or surface interface. This change in the refractive index is detected by varying the angle or wavelength at which the incident light is absorbed at the surface. The difference in the angle or wavelength is proportional to the amount of material bound on the surface, giving rise to a signal that is termed surface plasmon resonance (SPR), as discussed previously. See, for example, Nelson et al., Analytical Chemistry 71 (1999): 2858-2865; see also Nedelkov & Nelson, Biosensors and Bioelectronics 16 (2001): 1071-1078.

The SPR biosensing technology has been combined with MALDI-TOF mass spectrometry for the desorption and identification of biomolecules. In a chip-based approach to BIA-MS, a ligand , e.g., an antibody, is covalently immobilized on the surface of a chip. A tryptic digest of solubilized proteins from a sample is routed over the chip, and the relevant peptides or polypeptides, are bound by the ligand. After a washing step, the eluted peptides are analyzed by MALDI-TOF mass spectrometry. The system may be a fully automated process and is applicable to detecting and characterizing proteins present in complex biological fluids and cell extracts at low- to sub-femtomol levels.

Mass spectrometers useful for such applications are available from Applied Biosystems (Foster City, Calif.); Bruker Daltronics (Billerica, Mass.) and Amersham Pharmacia (Sunnyvale, Calif.).

Other suitable techniques for use in the present invention include “Multidimensional Protein Identification Technologies.” Cells are fractionally solubilized and digested, e.g., sequentially with endoproteinase Lys-C and immobilized trypsin. The samples are then subjected to multidimensional protein identification technology (MUDPIT), which involves a sequential separation of the peptide fragments by on-line biphasic microcapillary chromatography (e.g., strong ion exchange, then C-18 separation), followed by tandem mass spectrometry (MS-MS). See, for example, Washburn et al., Nature Biotechnology 19:242-247 (2001).

Articles of Manufacture

The invention also provides articles of manufacture. Articles of manufacture can include at least one oligonucleotide, as well as instructions for collecting a sample from grain and related products and using the oligonucleotide(s) to determine the presence, absence, or amount of microbial contamination in a sample from grain or a food product. An embodiment useful in an article of manufacture of the invention can be an oligonucleotide that is complementary to nucleic acid sequences from only one or a restricted number of microbial species, or can be an oligonucleotide that is complementary to nucleic acid sequences from more than one microbial species or across genera. A restricted number of species can refer to closely related species within a genus, or can refer to particular microorganisms that might be present in a particular sample (e.g., target organisms).

In one embodiment, the oligonucleotide(s) are attached to a microarray (e.g., a GeneChip®, Affymetrix, Santa Clara, Calif.). In another embodiment, an article of manufacture can include one or more oligonucleotide primers and one or more oligonucleotide probes. Such primers and probes can be used, for example, in real-time amplification reactions to amplify and simultaneously detect amplification products.

Suitable oligonucleotides include those that are complementary to highly conserved regions of a nucleic acid sequence and that flank a variable region. Such universal primers can be used to specifically amplify these variable regions, thereby providing a sequence with which to identify microorganisms. Examples of oligonucleotide primers, which are universal primers to microbial cpn60 sequences, include the following:

| | |
|---|---|
| 5′-GAIIIIGCIGGIGA(T/C)GGIACIA | (SEQ ID NO: 1) |
| CIAC-3′; and | |
| 5′-(T/C)(T/G)I(T/C)(T/G)ITCIC | (SEQ ID NO: 2) |
| C(A/G)AAICCIGGIGC(T/C)TT-3′. | |

Suitable oligonucleotides also include those that are complementary to species-specific sequences, and thus result in an amplification product only if a particular species is present in the sample.

Similar to oligonucleotide primers, oligonucleotide probes generally are complementary to the target sequences. Oligonucleotide probes can be designed to hybridize universally to target sequences, or can be designed for species-specific hybridization to a variable region of the target sequences.

An article of manufacture of the invention can further include additional components for carrying out amplification reactions and/or reactions, for example, on a microarray. Articles of manufacture for use with PCR reactions can include nucleotide triphosphates, an appropriate buffer, and a polymerase. An article of manufacture of the invention also can include appropriate reagents for detecting amplification products. For example, an article of manufacture can include one or more restriction enzymes for distinguishing amplification products from different species of microorganism, or can include fluorophore-labeled oligonucleotide probes for real-time detection of amplification products.

It will be appreciated by those of ordinary skill in the art that different articles of manufacture can be provided to evaluate microbes from different types of grain or related products. For example, wheat may contain a different community of microbes than that of corn. Therefore, an article of manufacture designed to evaluate the microbes in wheat may have a different set of controls or a different set of species-specific hybridization probes than that designed for use with corn. Alternatively, a more generalized article of manufacture can be used to evaluate the microbes in a number of different grain and related products.

In addition, articles of manufacture are provided that include at least one microbial antibody, as well as instructions for collecting a sample from grain or a related product and using the antibody to detect the presence, absence, or amount of microbial contamination in the grain or related product.

In one embodiment, one or more microbial antibodies are attached to a microarray (e.g., a 96-microwell plate). For example, a microarray format may include a variety of universal and specific capture antibodies; the universal and specific antibodies may each be located at a different well location. The article of manufacture may also include the appropriate detection antibodies, if necessary, and appropriate reagents for binding of a microbial polypeptide to one or more capture antibodies (e.g., enzymes, substrates, buffers, and controls).

In another embodiment, an article of manufacture can include one or more microbial antibodies attached to a dipstick. Such dipsticks can be used, for example, to detect microbial polypeptides in a liquid sample.

The invention will be further described in the following examples, which do not limit the scope of the invention described in the claims.

### EXAMPLES

### Example 1

### Sampling and Preparation of Grain

The recommended sampling procedure for detecting microbial contamination is to take at least ten samples and collect a total of at least 10 lbs of corn while its being transported in farm trucks or semi-loads. The corn must be below 16% moisture unless the test will be conducted and results received immediately. Delayed test results on high moisture corn may not be accurate because any fungus present can continue to grow and produce mycotoxins.

The recommended procedure is to grind the grain sample fine enough to pass through a number 14 sieve (about the consistency of sand) and then thoroughly mix it. A two to four lb. sub-sample is then ground so that it can pass through a No. 20 sieve (about the consistency of instant coffee) before testing for microbial nucleic acids and/or polypeptides. Grinding is done with a blender or coffee grinder. Grinders designed especially for grain provide the best results and durability. The sample is stored below 13% moisture to reduce and prevent the continued development of microbes.

### Example 2

### Testing Grain for Microbial Contamination

10 samples of corn totaling approximately 10 lbs. are obtained from a grain elevator. The sample is macerated and serially diluted in phosphate buffer (pH 7.0). The dilutions are plated onto plates containing plate count agar medium. The plates are grown at 37° C. overnight, and evaluated for microbial growth. The plates are left at 37° C. for an additional 24-48 hours and evaluated daily for microbial growth. The number of colonies from each dilution sample is counted with a plate reader after 48 hours of growth. In addition, the dilution samples are also plated onto brilliant green agar to detect any Salmonella spp. present in the sample. Brilliant green agar plates are incubated aerobically at 37° C. for 24 to 48 hours. Red colonies are presumptive Salmonella spp. If low numbers of colonies are observed, the undiluted sample is enriched in selenite cystine broth overnight, followed by plating on brilliant green agar. The dilution samples are additionally plated on MacConkey agar to detect the presence of E. coli and grown aerobically at 37° C. for 24 to 48 hours. Colony numbers on each plate are counted using a plate reader.

### Example 3

### Quantitating Microbial Organisms Using cpn60 Universal Primers and a cpn60 Universal Probe

A biological sample is obtained from a grain transport truck and genomic DNA is extracted using standard methods (Diagnostic Molecular Microbiology: Principles and Applications (supra)). Real-time PCR is conducted using universal cpn60 primers having the nucleotide sequences set forth in SEQ ID NO:1 and SEQ ID NO:2, and a universal cpn60 probe having the sequence 5′-GACAAAGTCGGTAAAGAAGGCGTTATCA-3′ (SEQ ID NO:3), labeled at the 5′ end with fluorescein (fluorophore; Molecular Probes, Inc.) and at the 3′ end with dabcyl (quencher; (4-(4′-dimethylaminophenylazo)benzoic acid) succinimidyl ester; Molecular Probes, Inc.). This probe binds to a variable region of the cpn60 gene from numerous microbial species; thus the Tm of the probe from an amplification product varies depending upon the nucleotide sequence within the amplification product to which the probe hybridizes.

The PCR reaction contains 3 μL extracted DNA, 1 μM each universal cpn60 primer, 340 nM universal cpn60 probe, 2.5 units Amplitaq Gold DNA polymerase (Perkin Elmer), 0.25 mM each deoxyribonucleotide, 3.5 mM MgCl₂, 50 mM KCl, and 10 mM Tris-HCl, pH 8.0 in a total reaction volume of 50 μL. PCR conditions include an initial incubation at 95° C. for 10 minutes to activate the Amplitaq Gold DNA polymerase, followed by 40 cycles of 30 seconds at 95° C., 60 seconds at 50° C., and 30 seconds at 72° C. Fluorescence is monitored during the 50° C. annealing steps throughout the 40 cycles. After the cycling steps are complete, the melting temperature of the universal probe from the amplification products is analyzed. As the temperature is increased, the universal probe is released from the amplification product from each species' cpn60 sequence at a specific temperature, corresponding to the Tm of the universal probe and the cpn60 sequence of the particular species. The step-wise loss of fluorescence at particular temperatures is used to identify the particular species present, and the loss in fluorescence of each step compared to the total amount of fluorescence correlates with the relative amount of each microorganism.

### Example 4

### Dipstick ELISA Assay for Staphylococcus

A polystyrene dipstick containing two horizontal bands is constructed: one band consists of broadly reactive, polyclonal capture antibodies against cpn60 proteins from Staphylococcus spp., while the other band is an internal control consisting of horseradish peroxidase. The assay is performed by making serial dilutions (1:2, 1:5, 1:10, etc.) of a grain sample that has been prepared as described above in Example 1. The dilutions are added directly into a detection reagent and incubating a wetted dipstick in these dilutions for 5 minutes, and then adding an indicator to detect binding of cpn60 proteins to the capture (and detection) antibodies. The detection reagent includes a suitable buffer and secondary cpn60 Staphylococcus detection antibodies labeled with horseradish peroxidase. The indicator can be a chromogenic horseradish peroxidase substrate, such as 2,2′-AZINO-bis 3-ethylbenziazoline-6-sulfonic acid, or ABTS. ABTS is considered a safe, sensitive substrate for horseradish peroxidase that produces a blue-green color upon enzymatic activity that can be quantitated at 405-410 nm. At the end of the incubation and indicator steps, the dipstick is rinsed with water (e.g., deionized water) and examined for staining of the antibody band by visual inspection. Staining of the antibody band reveals the presence of Staphylococcus spp. in the sample. The internal control band provides a check on the integrity of the detection reagent.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining the presence, absence, or amount of microbial contamination in grain or a related product, comprising:
providing a sample from said grain or related product; and
determining the presence, absence, or amount of microbial contamination in said sample,
wherein the presence, absence, or amount of microbial contamination in said sample indicates the presence, absence, or amount, respectively, of microbial contamination in said grain or related product.

2. The method of claim 1, wherein microbes are taxonomically and phylogenetically identified.

3. The method of claim 2, wherein said microbes are selected from the group consisting of bacteria, fungi, viruses, and protozoa.

4. The method of claim 3, wherein said bacterial microbe is selected from the group consisting of the Staphylococcus, Streptococcus, Pseudomonas, Escherichia, Bacillus, Brucella, Chlamydia, Clostridium, Shigella, Mycobacterium, Agrobacterium, Bartonella, Borellia, Bradyrhizobium, Ehrlichia, Haemophilus, Helicobacter, Heliobacter, Lactobacillus, Neisseria, Rhizobium, Streptomyces, Synechococcus, Zymomonas, Synechocyotis, Mycoplasma, Yersinia, Vibrio, Burkholderia, Franciscella, Legionella, Salmonella, Bifidobacterium, Enterococcus, Enterobacter, Citrobacter, Bacteroides, Prevotella, Xanthomonas, Xylella, and Campylobacter genera.

5. The method of claim 3, wherein said fungal microbe is selected from the group consisting of the Aspergillus, Fusarium, Penicillium, Claviceps, Colletrotrichum, Cochliobolus, Helminthosporium, Microcyclus, Puccinia, Pyricularia, Deuterophoma, Monilia, Candida, and Saccharomyces genera.

6. The method of claim 5, further comprising:
performing an enzyme-linked immunosorbent assay (ELISA) on a sample from said grain or related product, wherein said ELISA is specific for mycotoxin produced by said fungal microbe.

7. The method of claim 3, wherein said viral microbe is from the Coronaviridae genus.

8. The method of claim 3, wherein said protozoan microbe is selected from the group consisting of the Acanthamoeba, Cryptosporidium, and Tetrahymena genera.

9. The method of claim 1, wherein said grain is selected from the group consisting of corn, wheat, barley, sorghum, and rice.

10. The method of claim 1, wherein said grain is processed grain.

11. The method of claim 1, wherein said related product is selected from the group consisting of rice, breads, muffins, cakes, cereals, pasta, and dough.

12. The method of claim 1, wherein said sample is a plurality of samples.

13. The method of claim 12, wherein said plurality of samples are pooled.

14. The method of claim 1, wherein said determining comprises microbial culturing and colony identification.

15. The method of claim 1, wherein said determining comprises genetic fingerprinting.

16. The method of claim 1, wherein said determining comprises ribosomal genotyping.

17. The method of claim 1, wherein said determining comprises cpn60 genotyping.

18. The method of claim 1, further comprising:
providing a control sample; and
determining the amount of microbes in said control sample.

19. The method of claim 18, wherein said control sample is a known amount of microbes.

20. A method for monitoring grain or a related product before, during, or after processing of said grain or said related product, comprising:
providing a sample from said grain or related product;
determining the presence, absence, or amount of microbial contamination in said sample; and
tailoring said processing based upon said presence, absence, or amount of said microbial contamination.

21. The method of claim 20, wherein when said sample is obtained before said processing of said grain into said related product, said tailoring comprises:
abandoning said processing; or
expediting said processing.

22. The method of claim 20, wherein when said sample is obtained during said processing of said grain into said related product, said tailoring comprises:
abandoning said processing;
expediting said processing; or
repeating all or a portion of said processing.

23. The method of claim 20, wherein when said sample is obtained after said processing of said grain into said related product, said tailoring comprises:
repeating all or a portion of said processing.

24. The method of claim 20, wherein said grain is processed into animal feed.

25. The method of claim 20, wherein said tailoring is based on the presence or absence of said microbial contamination.

26. The method of claim 20, wherein said tailoring is based on the amount of said microbial contamination.

27. The method of claim 20, wherein said determining is specific for only one or a restricted number of microbial species.

28. The method of claim 20, wherein said microbial contamination is by only one or a restricted number of microbial species.

29. The method of claim 20, wherein said determining is for microbial species of at least two genera.

30. The method of claim 20, wherein said microbial contamination is by microbial species of at least two genera.

31. A method for monitoring grain or animal feed, comprising:
providing a sample from said grain or feed;
determining the presence, absence, or amount of microbial contamination in said sample; and
tailoring treatment of said grain or animal feed based upon said presence, absence, or amount of said microbial contamination.

32. The method of claim 31, wherein said treatment comprises blending said grain or said feed with grain or feed, respectively, that lacks microbial contamination.

33. The method of claim 31, wherein said treatment comprises feeding select animals with said animal feed.

34. The method of claim 31, wherein said treatment comprises applying a mycotoxin binder to said animal feed.

35. The method of claim 31, wherein said treatment comprises applying an acid-based fungal inhibitor to said grain.

36. An article of manufacture, comprising:
at least one anti-microbial antibody, wherein said antibody is attached to a solid support; and
instructions for collecting a sample from grain or a related product and determining the presence, absence, or amount of microbial contamination in said sample.

37. The article of manufacture of claim 36, further comprising an indicator molecule.

38. The article of manufacture of claim 36, wherein said solid support is a dipstick.

39. An article of manufacture, comprising:
at least one oligonucleotide, wherein said oligonucleotide is complementary to nucleic acid sequences from only one or a restricted number of microbial species; and
instructions for collecting a sample from grain or a related product and determining the presence, absence, or amount of microbial contamination in said sample.

40. An article of manufacture, comprising:
at least one oligonucleotide, wherein said oligonucleotide is complementary to nucleic acid sequences from microbial species of at least two genera; and
instructions for collecting a sample from grain or a related product and determining the presence, absence, or amount of microbial contamination in said sample.
